(19) United States
(12) Patent Application Publication
(10) Pub. No.: US 2017/0182325 A1
(43) Pub. Date: Jun. 29, 2017

(54) CASE STRUCTURE FOR IMPLANTABLE ELECTRONIC DEVICE

(71) Applicant: GiMer Medical Co., Ltd., New Taipei City (TW)

(72) Inventor: Chen-Tun WU, New Taipei City (TW)

(21) Appl. No.: 15/392,871

(22) Filed: Dec. 28, 2016

(30) Foreign Application Priority Data
Dec. 28, 2015 (CN) 201510993708.6

(51) Int. Cl. A61N 1/375

(52) U.S. Cl. CPC A61N 1/375

(57) ABSTRACT

A case structure for accommodating a circuit board of an implantable electronic device includes a housing and a lid body. The housing has a plate portion, a wall portion and an opening. One end of the wall portion connects to the periphery of the plate portion, and the other end of the wall portion defines the opening. The periphery of the plate body is sealed with the other end of the wall portion. Accordingly, the plate body and the housing together form an airtight space for accommodating the circuit board.

### CROSS REFERENCE TO RELATED APPLICATIONS

This Non-provisional application claims priority under 35 U.S.C. §119(a) on Patent Application No(s). 201510993708.6 filed in People's Republic of China on Dec. 28, 2015, the entire contents of which are hereby incorporated by reference.

### BACKGROUND OF THE INVENTION

Field of Invention

The present invention relates to a case structure for an implantable electronic device and, in particular, to a case structure being benefit for supersonic welding technology.

Related Art

Today, the technology of precision micro processing has matured to the point where it is possible to miniaturize a medical instrument (e.g. an implantable electronic device) to be implantable into an organism for a proper therapy. The implantable electronic device is, for example, an implantable neural electrical stimulator, a blood glucose sensor, or an artificial pacemaker. However, in view of the need to implant into the human body, researchers must find a way to effectively seal the case for the implantable electronic device in order to prevent body fluids from penetrating into the implantable electronic device and causing the short circuit or damage of the electronic device.

The conventional way is to use the adhesive glue for sealing the case which is made of plastic. However, the adhesive glue is easy to degrade because of the change of temperature. At the same time, the degraded adhesive glue may produce toxicity to the body. In addition, the adhesive way is slow and the production capacity is low, which will make the cost higher. Therefore, the technology of ultrasonic welding, which is fast, non-toxic and excellent sealing, becomes the best choice for packaging the implantable electronic devices with plastic cases.

The theory of the supersonic welding is to generate a high-frequency signal (generally between 20 KHz and 15 KHz). Then, a horn is direction contacted with the plastic work piece for transmitting the high-frequency signal to the plastic work piece. Accordingly, the molecules of the work piece are vibrated and fractioned so as to generate heat for melting the plastic work piece, thereby achieving the welding effect.

FIG. 1A is a schematic diagram showing a conventional case structure for an implantable electronic device, FIG. 1B is an exploded view of the case structure of FIG. 1A, and FIG. 1C is a schematic diagram showing a part of the case structure of FIG. 1A as performing the supersonic welding operation to the line A-A. Referring to FIGS. 1A to 1C, the implantable electronic device 1 includes a circuit board P and a case structure 10. The circuit board P is disposed in the case structure 10. As shown in FIG. 1B, the conventional case structure 10 has an upper lid 12 and a lower lid 14 corresponding to each other. Since the intensity of the high-frequency signal attenuates with the square of the distance, the energy of the high-frequency signal provided by the supersonic machine will attenuate if the upper lid has thicker thickness (the distance d between the welding surface D and the horn H). In this case, the processing time of the welding becomes longer and the power consumption is also higher.

Therefore, it is an important subject to provide a case structure for an implantable electronic device that can effectively control the energy transmission during the packaging process by the supersonic welding technology, thereby improving the welding speed and quality.

### SUMMARY OF THE INVENTION

In view of the foregoing, an objective of the present invention is to provide a case structure for an implantable electronic device that can effectively control the energy transmission during the packaging process by the supersonic welding technology, thereby improving the welding speed and quality.

To achieve the above objective, the present invention discloses a case structure for accommodating a circuit board of an implantable electronic device. The case structure includes a housing and a plate body. The housing has a plate portion, a wall portion and an opening. One end of the wall portion connects to a periphery of the plate portion, and the other end of the wall portion defines the opening. The plate body has a periphery sealed with the other end of the wall portion. The plate body and the housing together form an airtight space for accommodating the circuit board.

In one embodiment, the wall portion has a height of 5˜10 mm.

In one embodiment, a ratio of a thickness of the plate body and a height of the wall portion is between 0.04 and 0.4.

In one embodiment, the wall portion is sealed with the plate body by ultrasonic welding.

In one embodiment, the housing and the plate body are made of polyetheretherketone (PEEK).

In one embodiment, the housing has a positioning member disposed at a connection of the plate portion and the wall portion for fixing the circuit board.

In one embodiment, the wall portion has at least two through holes.

The present invention can decrease the distance between the welding surface and the horn of supersonic welding, thereby sufficiently improving the transmission performance of the high-frequency signal for performing the supersonic welding and enhancing the speed and quality of supersonic welding.

### BRIEF DESCRIPTION OF THE DRAWINGS

The invention will become more fully understood from the detailed description and accompanying drawings, which are given for illustration only, and thus are not limitative of the present invention, and wherein:

FIG. 1A is a schematic diagram showing a conventional case structure for an implantable electronic device;

FIG. 1B is an exploded view of the case structure of FIG. 1A;

FIG. 1C is a schematic diagram showing a part of the case structure of FIG. 1A as performing the supersonic welding operation to the line A-A;

FIG. 2 is a schematic diagram showing a case structure of an implantable electronic device according to an embodiment of the invention;

FIG. 3A is a schematic diagram showing an assembled case structure of an implantable electronic device according to an embodiment of the invention;

FIG. 3B is a sectional view along the line B-B of FIG. 3A;

FIG. 3C is a sectional view of the C area of FIG. 3B before sealing the case structure by supersonic welding; and

FIG. 3D is a sectional view of the C area of FIG. 3B after sealing the case structure by supersonic welding.

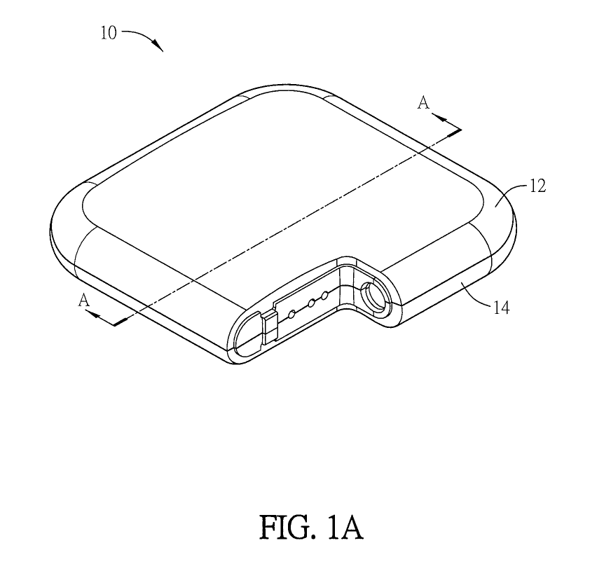

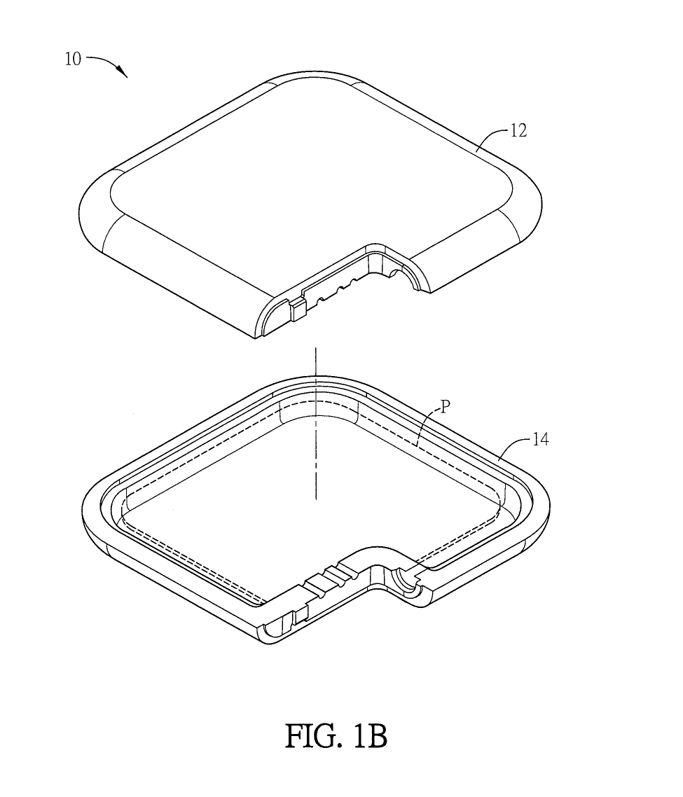

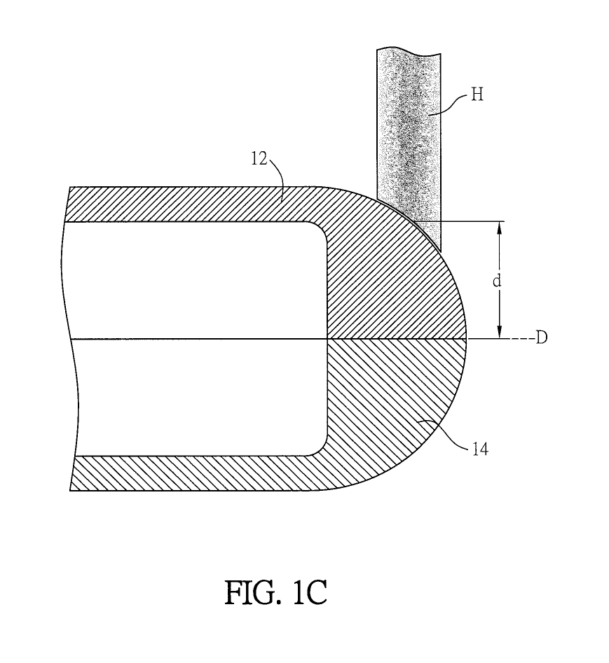

### DETAILED DESCRIPTION OF THE INVENTION

The present invention will be apparent from the following detailed description, which proceeds with reference to the accompanying drawings, wherein the same references relate to the same elements.

To be noted, the implantable electronic device is a medical electronic device to be implanted in a body, such as an implantable neural electrical stimulator, a blood glucose sensor, or an artificial pacemaker. The case structure of an implantable electronic device is not limited to any specific medical field. To make the following description more comprehensive, the implantable medical electronic device hereinbelow is an implantable neural electrical stimulator, which is used for treating or relieving the pain of an affected part of an organism. Herein, the mentioned organism can be the mouse, human, rabbit, cattle, sheep, pig, monkey, dog, cat and the likes, and preferably a human body.

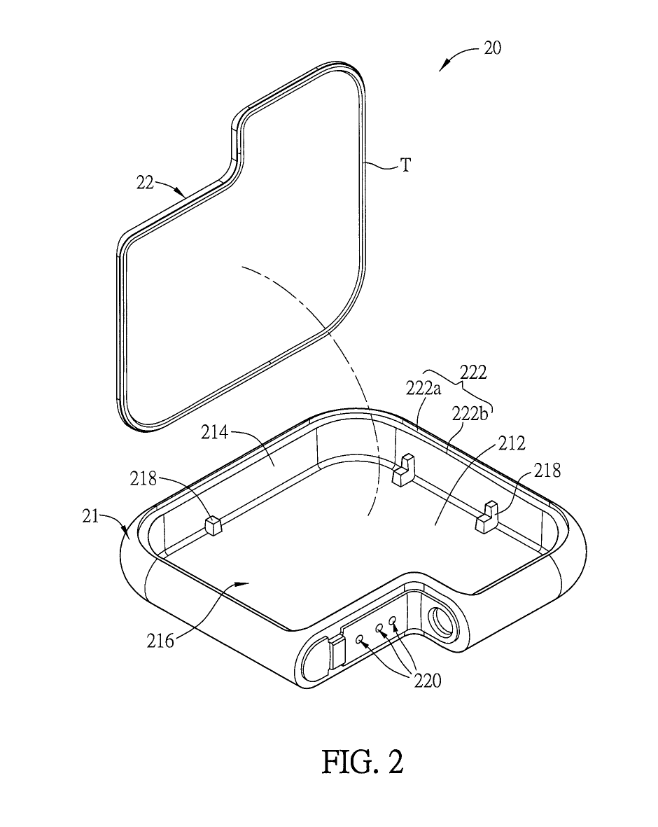

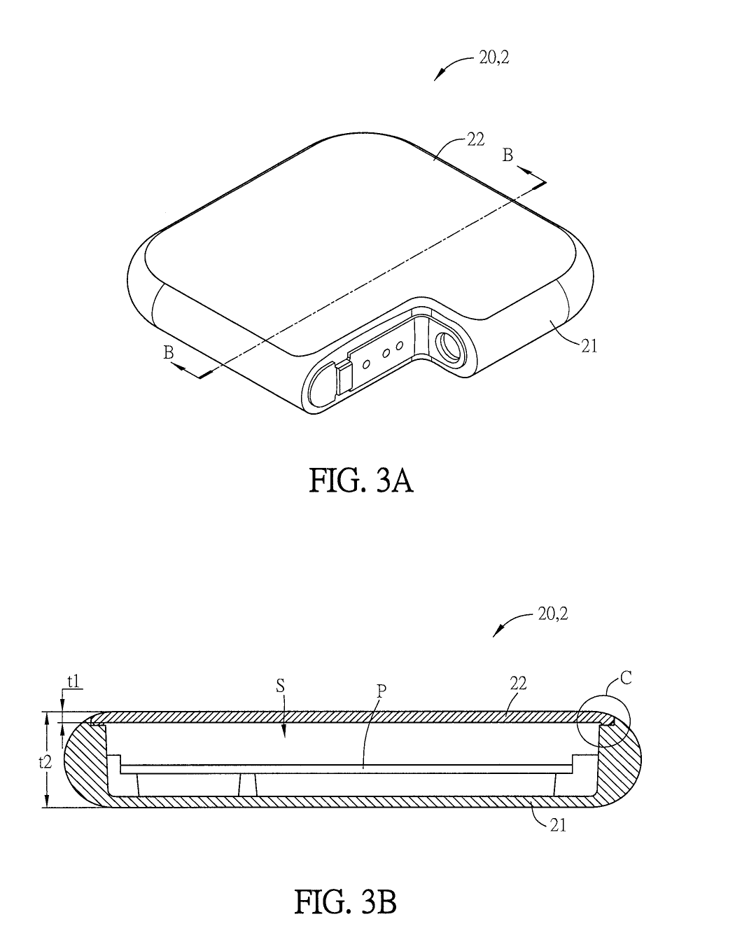

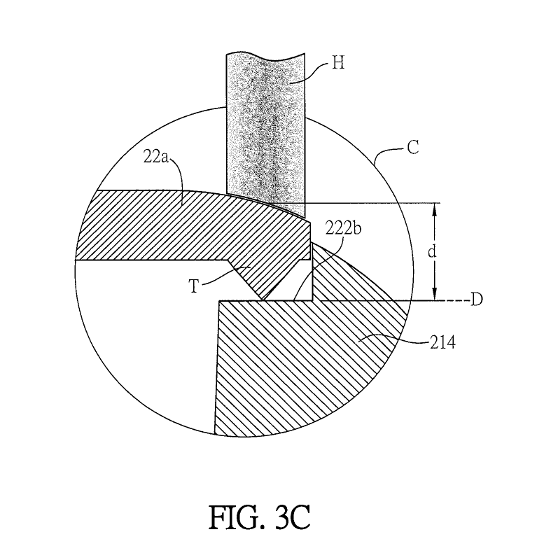

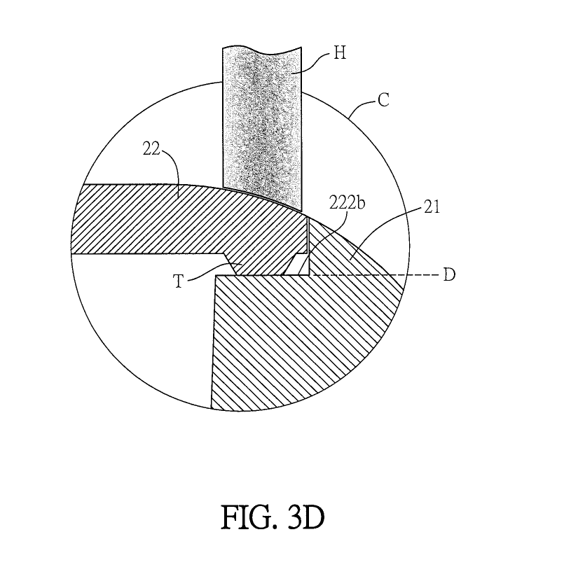

FIG. 2 is a schematic diagram showing a case structure of an implantable electronic device according to an embodiment of the invention. As shown in FIG. 2, the implantable electronic device 2 includes a case structure 20 and a circuit board P (as shown in FIG. 3B). The case structure 20 is configured to accommodate the circuit board P (as shown in FIG. 3B). The case structure 20 includes a housing 21 and a plate body 22. In this embodiment, the case structure 20 is made of polyetheretherketone (PEEK), which has good corrosion resistance and hydrolysis resistance. Since the case structure 20 can directly contact with the human body, the PEEK material can prevent the undesired harm to the human body. Besides, the PEEK material is a thermoplastic material, so it is suitable in the supersonic welding technology for sealing and connecting the housing 21 and the plate body 22.

The housing 21 includes a plate portion 212, a wall portion 214, an opening 216, and a plurality of positioning members 218. One end of the wall portion 214 connects to a periphery of the plate portion 212, and the body of the wall portion 214 extends from the periphery of the plate portion 212 upwardly. The other end of the wall portion 214 defines the opening 216. In this embodiment, the wall portion 214 is substantially perpendicular to the plate portion 212. To be noted, this configuration is for an illustration only and is not to limit the invention. In this embodiment, the wall portion 214 and the plate portion 212 are integrally formed as one piece. In other embodiments, the wall portion 214 and the plate portion 212 can be separated parts and are assembled by adhering or welding, and this invention is not limited. The other end of the wall portion 214 is configured with a recess portion 222, which has two contact surfaces 222a and 222b. The two contact surfaces 222a and 222b are substantially perpendicular to each other, and the recess portion 222 can fit and connect to the plate body 22.

FIG. 3A is a schematic diagram showing an assembled case structure of an implantable electronic device according to an embodiment of the invention, and FIG. 3B is a sectional view along the line B-B of FIG. 3A. Referring to FIGS. 2, 3A and 3B, the case structure 20 accommodates the circuit board P. As shown in FIGS. 3A and 3B, the housing 21 and the plate body 22 are connected to form a space S for accommodating the circuit board P. The circuit board P is disposed in the housing 21 through the opening 216. The wall portion 214 is configured with a plurality of through holes 220, and a plurality of conducting sheets or rods (not shown) are disposed through the through holes 220. The circuit board P is coupled to the connector through the conducting sheets or rods so as to electrically connect to the external medical device. Moreover, the external medical device can adjust or set up the parameters of the circuit board P. The positioning members 218 are distributed at the connection of the plate portion 212 and the wall portion 214. When the circuit board P is disposed in the space S, the positioning members 218 can contact with the circuit board P (see FIG. 3B) for fixing the circuit board P in the space S. This configuration can prevent the damage of the circuit board P caused by collision. To be noted, the circuit board P can be a printed circuit board, a flexible circuit board, or a control circuit board with IC. In addition, the space S may accommodate other necessary electronic devices.

FIG. 3C is a sectional view of the C area of FIG. 3B before sealing the case structure by supersonic welding, and FIG. 3D is a sectional view of the C area of FIG. 3B after sealing the case structure by supersonic welding. Referring to FIG. 3C, before the supersonic welding process, the plate body 22 has a micro protruding T for contacting with the contact surface 222b. During the supersonic welding process, the micro protruding T is melted. Thus, as shown in FIG. 3D, after the supersonic welding process, the micro protruding T is deformed and bonded with the contact surface 222b. As shown in FIG. 3C, a distance d between the welding surface D and the horn H is the distance for transmitting energy by supersonic wave, and the distance d is mostly determined by the thickness of the plate body 22. In the design of the thickness of the case structure is not uniform, so that the supersonic wave can be applied to the thinner part of the case structure for performing the supersonic welding. This can sufficiently reduce the attenuation of the high-frequency signal and thus improve the speed of supersonic welding.

To be noted, the case structure of the invention has a shorter distance d between the welding surface D and the horn H of the supersonic welding machine.

This design can decrease the energy loss of the supersonic wave, thereby sufficiently improving the performance and quality of the supersonic welding. In an embodiment as shown in FIG. 3B, the plate body 22 substantially does not have a wall portion. A ratio of the average thickness t1 of the plate body 22 to the height of the wall portion 214 is smaller than 1 and is preferably between 0.04 and 0.4. The height of the wall portion 214 is between 5 mm and 10 mm. Accordingly, the supersonic welding technology can achieve the optimum effect in this invention.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. A case structure for accommodating a circuit board of an implantable electronic device, comprising:
a housing having a plate portion, a wall portion and an opening, wherein one end of the wall portion connects to a periphery of the plate portion, and the other end of the wall portion defines the opening; and
a plate body having a periphery sealed with the other end of the wall portion, wherein the plate body and the housing together form an airtight space for accommodating the circuit board.

2. The case structure of claim 1, wherein the wall portion has a height of 5˜10 mm.

3. The case structure of claim 1, wherein a ratio of a thickness of the plate body and a height of the wall portion is between 0.04 and 0.4.

4. The case structure of claim 1, wherein the wall portion is sealed with the plate body by ultrasonic welding.

5. The case structure of claim 1, wherein the housing and the plate body are made of polyetheretherketone (PEEK).

6. The case structure of claim 1, wherein the housing has a positioning member disposed at a connection of the plate portion and the wall portion for fixing the circuit board.

7. The case structure of claim 1, wherein the wall portion has at least two through holes.

8. An implantable electronic device, comprising:
a housing having a plate portion, a wall portion and an opening, wherein one end of the wall portion connects to a periphery of the plate portion, and the other end of the wall portion defines the opening;
a plate body having a periphery sealed with the other end of the wall portion, wherein the plate body and the housing together form an airtight space; and
a circuit board accommodated in the airtight space.
